(19) United States
(12) Patent Application Publication
(10) Pub. No.: US 2009/0131979 A1
(43) Pub. Date: May 21, 2009

(54) Wound closure method apparatus and system

(76) Inventors: G. Alan Thompson, Ottawa (CA); Richard Edward Brois, SR., Raeford, NC (US); Christopher Matthew Scheuerman, SR., Sanford, NC (US); David Curry Pitts, JR., Spring Lake, NC (US)

Correspondence Address: BERGMAN & SONG, LLP, P.O. BOX 400198, CAMBRIDGE, MA, 02140, US

(21) Appl. No.: 12/218,981

(22) Filed: Jul. 17, 2008

Related U.S. Application Data
(60) Provisional application No. 60/959,960, filed on Jul. 17, 2007.

(51) Int. Cl. A61B 17/04; A61B 19/00

(52) U.S. Cl. 606/224; 606/228; 606/222; 128/898

(57) ABSTRACT

Disclosed is a wound closure device, such as a suture, including a substantially flexible tensile member, the tensile member having an external surface, the external surface including a biocidal material such as a coating of substantially pure silver. A method of applying the suture includes piercing a patient's skin adjacent to a wound using, for example, a needle and drawing a substantially flexible tensile member through a resulting aperture in the skin. By virtue of the biocidal material, pathogens and other bioactive materials drawn through the aperture are rendered less active and/or incapable of causing infection.

### CROSS-REFERENCE TO RELATED APPLICATIONS

The present application claims the benefit of U.S. Provisional patent application No. 60/959,960 filed on Jul. 17, 2007, the disclosure of which is incorporated herewith in its entirety.

### FIELD OF THE INVENTION

The present invention relates to wound closure devices and more particularly to wound closure devices having biocidal properties.

### BACKGROUND

It has long been understood that any breach of the skin offers an opportunity for the entry of microbes into a body, and a consequent risk of infection. Consequently, for as long as the germ theory of disease has been accepted, first-aid givers and other medical practitioners have attempted to avoid the introduction of contaminants into a wound, and to ameliorate the effects of wound contamination. These efforts have included the application of topical cleansers, antiseptics, and antibiotics to wounds, and to surrounding skin, the localized injection of antimicrobial agents including antibiotics, and the systemic application of medicines, again including antibiotics. In addition, efforts have been made to provide sterile wound dressings and ancillary materials, as well as sterile instruments, and clothing, including sterile gloves.

Despite this effort, the contamination of wounds with biological agents remains a persistent and important problem. An important subset of this contamination problem surrounds the use of wound closure devices. When the skin is breached, either intentionally by a surgical incision, or unintentionally, as in the case of trauma, it is often necessary to use mechanical closures to close the breach. Often, these mechanical closures include sutures, ligatures, and/or surgical staples. Generally speaking, these mechanical closures operate by piercing skin and flesh adjacent to a wound or incision and applying tensile forces to pull or retain opposite edges of the wound into proximity with one another.

The process of piercing the skin and flesh to apply a mechanical closure offers a further opportunity, in addition to the wound itself, for the introduction of pathogens and other biological agents into the body of the patient. Consequently, an effort to close a wound, and thereby aid healing and exclude biological agents, may result in the contrary effect of introducing adverse biological agents. As noted above, previous efforts to avoid such contamination have included the application of antiseptic materials adjacent to a wound prior to the installation of sutures or other closures, the pre-sterilization of closure devices, and the prophylactic and remedial administration of antibiotic substances. Nevertheless, and in spite of long and concerted efforts to solve these problems, the problem of contamination of mechanical wound closure sites, and consequent infection, persists.

### SUMMARY

Being aware of the long, and previously incompletely effective efforts of others to address these problems, the present inventors have arrived at a new understanding of the problem of infection related to wound closure mechanisms, and have conceived, and do here present, novel and effective solutions to these problems. In particular, it is understood that earlier efforts to sterilize the skin surrounding a wound have been inconsistently effective. This is true in a substantially sterile surgical environment, and even more true in the application of emergent medicine (i.e., first aid) to, for example, trauma victims.

The inventors have developed an important understanding that, particularly in military combat environments, where trauma is often severe and where treatment time may be constrained by ongoing fighting, it is very difficult to achieve sufficiently sterile skin conditions to avoid the introduction of infective agents through the skin during application of wound closure mechanisms (e.g., suturing).

Having developed this fundamental understanding, the inventors have created and developed wound closure mechanisms including biocidal agents adapted to suppress or destroy the activity of pathogens which are necessarily introduced into a patient's body during application of the wound closure mechanisms. Therefore, the present specification and claims disclose novel wound closure mechanisms including surgical ligatures, sutures and staples, including biocidal materials incorporated therewithin and thereupon. In various embodiments, the invention includes a suture having a metallic silver coating deposited on an external surface thereof. In certain embodiments, this silver surface coating extends inwardly of the surface into a region proximate to the surface, up to and including throughout the bulk of the material.

These and other advantages and features of the invention will be more readily understood in relation to the following detailed description of the invention, which is provided in conjunction with the accompanying drawings.

### BRIEF DESCRIPTION OF THE DRAWINGS

FIG. 1 shows a wound closure device configured as a suture according to principles of the invention;

FIGS. 2A-2G show various profiles of longitudinal portions according to principles of the invention;

FIG. 3 shows a manufacturing fixturing arrangement according to principles of the invention;

FIG. 4 shows a wound closure device configured as a surgical staple according to principles of the invention;

FIG. 5 shows a wound closure device configured as a surgical staple according to principles of the invention;

FIG. 6 shows exemplary packaging for a wound closure device according to principles of the invention;

FIG. 7 shows exemplary packaging for a wound closure device according to principles of the invention; and

FIG. 8 shows a wound closure device applied to a wound on an arm of an exemplary patient according to principles of the invention.

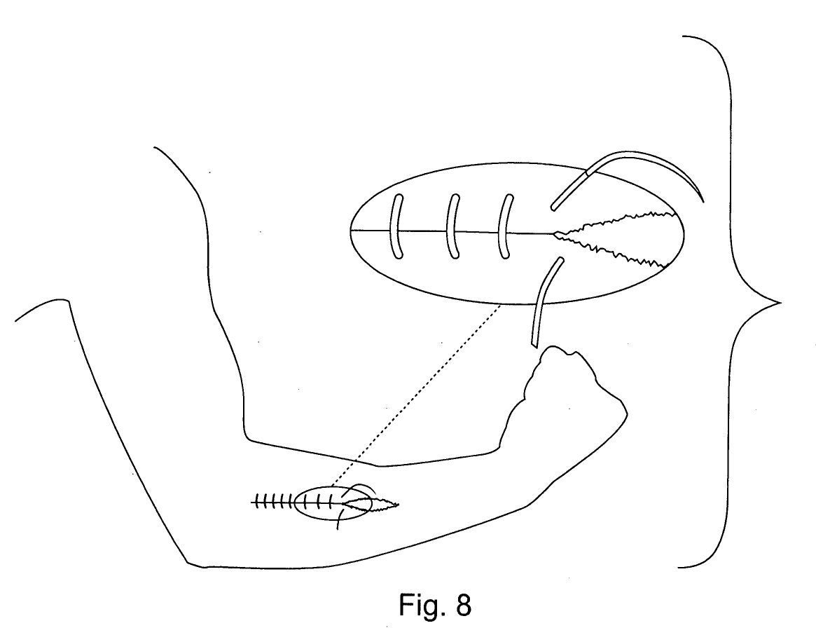

### DETAILED DESCRIPTION

The following description is provided to enable any person skilled in the art to make and use the disclosed inventions and sets forth the best modes presently contemplated by the inventors of carrying out their inventions. In the following description, for purposes of explanation, numerous specific details are set forth in order to provide a thorough understanding of the present invention. It will be apparent, however, to one skilled in the art that the present invention may be practiced without these specific details. In other instances, well-known structures and devices are shown in schematic form in order to avoid unnecessarily obscuring the present invention.

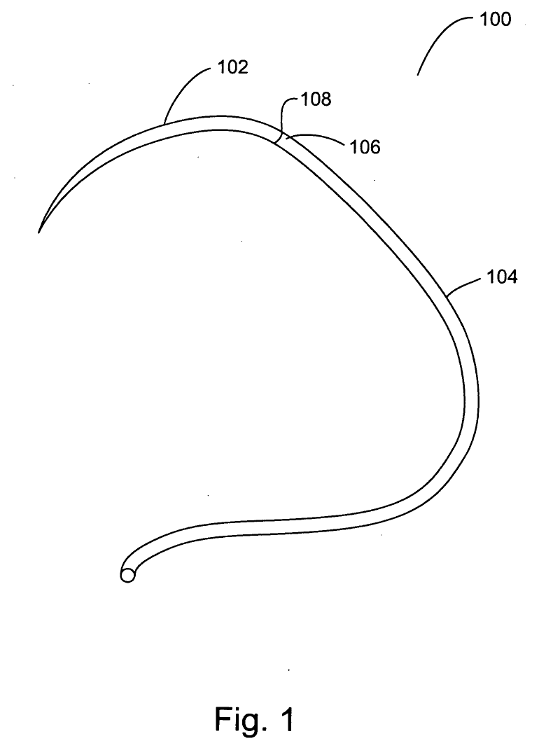

FIG. 1 shows a first embodiment of the invention. In the first embodiment, a suture device 100 includes a substantially rigid needle portion 102 and a substantially flexible longitudinal portion 104. The longitudinal portion 104 has a first end 106 substantially fixedly coupled to a second end 108 of the needle portion 102. In various embodiments, the longitudinal portion includes one or more of a natural polymer material, a synthetic polymer material, and a metallic material. In certain embodiments, the natural polymer serial includes a natural silk material. In other embodiments, the synthetic polymer material includes a polyamide material, a polyaramid material, a polybutylene material; an acrylonitrile butadiene styrene (ABS) polymer material, a polypropylene material, a polyvinyl chloride material, a polyester material, or other synthetic or natural polymer material, including combinations thereof, as known in the art.

According to one embodiment of the invention, the substantially rigid needle portion 102 is disposed in an arcuate arrangement (as illustrated). In another embodiment of the invention, the needle portion 102 is disposed in a substantially linear arrangement. According to one embodiment, the needle portion 102 includes a region having a cutting profile, as is known in the art. In another embodiment, the needle portion 102 includes a non-cutting profile. According to one embodiment, the needle portion 102 includes a stainless steel material including, for example, a surgical stainless steel. According to a further embodiment of the invention, the needle portion 102 includes a substantially rigid polymer member. In various embodiments, the substantially rigid polymer member includes a polyamide material, a polyaramid material, a polybutylene material; an acrylonitrile butadiene styrene (ABS) polymer material, a polypropylene material, a polyvinyl chloride material, a polyester material, or other synthetic or natural polymer material, including combinations thereof, as known in the art.

According to certain embodiments of the invention, the polymer material includes a reinforcing material, or filler. In various embodiments, the reinforcing material includes one or more of carbon, silicon, and a metallic material. In certain embodiments of the invention, the reinforcing material includes a carbon fiber. In other embodiments of the invention, the reinforcing material includes a glass fiber material and in still other embodiments of the invention, the reinforcing material includes a metallic fiber material. In certain embodiments of the invention, the reinforcing material includes macroscopic scale fibers and/or particles. In other embodiments of the invention, the reinforcing material includes microscopic scale fibers and/or particles and in still other embodiments of the invention, the reinforcing material includes nanoscale fibers and/or particles.

In one embodiment of the invention, the substantially flexible longitudinal portion 104 includes a monofilament fiber portion. According to one embodiment, the monofilament portion has a substantially circular profile. In another embodiment of the invention, the monofilament portion has a substantially elliptical profile. In yet another embodiment of the invention, the monofilament portion has a substantially symmetrically grooved profile. In yet another embodiment of the invention, the monofilament portion has an asymmetrically grooved profile. In one embodiment, the grooved profile includes a deep groove. In another embodiment, the grooved profile includes a shallow groove. In still another embodiment, a combination of both deep and shallow grooves are present in the profile.

In one embodiment of the invention, a deep groove includes a groove having an aspect ratio of from at least about 50% to at least about 10%, where the aspect ratio divides an average width of the groove by an average depth of the groove. In another embodiment of the invention, a deep groove includes a groove having an aspect ratio of from at least about 60% to at least about 1%. In certain embodiments of the invention, a surface of the groove is substantially silverized throughout the depth of the groove. In certain embodiments, high capillary action is achieved by the presence of the deep groove with respect to intrinsic and/or externally introduced fluids.

In a further embodiment of the invention, the longitudinal portion 104 includes a multi-stranded fiber portion. In still another embodiment of the invention, the longitudinal portion 104 includes a portion incorporating a composite of microfibers, and in still another embodiment, the longitudinal portion 104 includes a plurality of surface microfibers forming a high-surface-area nap. In certain embodiments of the invention, the micro-fibers of the high surface area nap include up substantially uniformly silverized surface region.

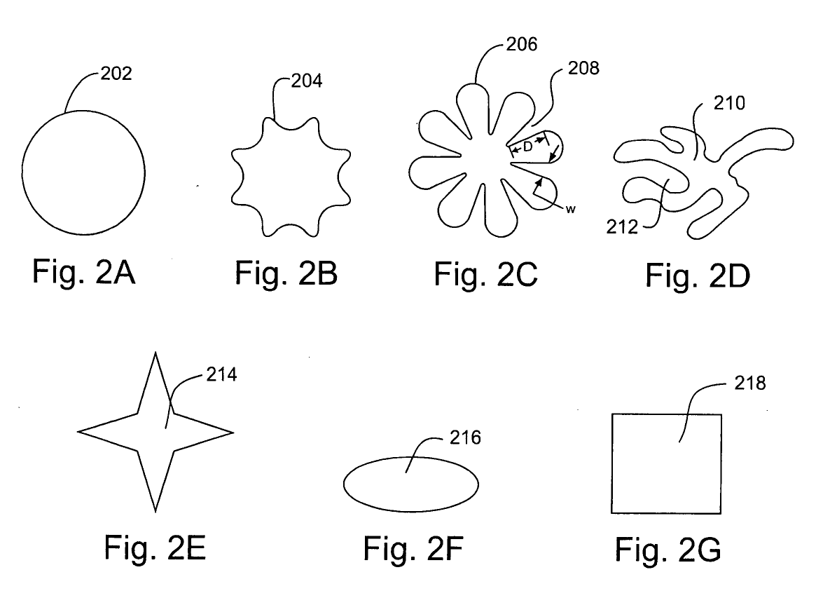

FIGS. 2A-2G show various cross-sectional fiber or suture profiles, according to respective embodiments of the invention. For example, FIG. 2A shows a cross-section 202 of a region of a fiber having a substantially circular profile. FIG. 2B shows a cross-section 204 of a region of a fiber having a shallow-grooved profile. FIG. 2C shows a cross-section 206 of a region of a fiber having a plurality of substantially symmetrical deep grooves, including groove 208. FIG. 2D show a cross-section 210 of a region of a fiber having substantially asymmetrical deep grooves 212. FIG. 2E shows a cross-section 214 of a region of a fiber having a substantially stellate perimeter. FIG. 2F shows a cross-section 216 of a region of a fiber having a substantially elliptical perimeter, and FIG. 2G shows a cross-section 218 of a fiber having a substantially rectangular perimeter. In the illustrated embodiment, the substantially rectangular perimeter is shown as an exemplary substantially square perimeter.

It should be noted that, while the examples above have been discussed in relation to sutures, one of skill in the art will appreciate that, in other embodiments, the same forms, materials and principles are readily adapted for use in ligatures and other wound closing devices.

Referring again to FIG. 1, in one embodiment of the invention, substantially flexible longitudinal portion 104 includes a polyamide material, and in another embodiment a polyaramid material. In still another embodiment, longitudinal portion 104 includes a polyester material, and in various other embodiments, longitudinal portion 104 includes natural and synthetic polymer materials such as would be known to one of skill in the art.

According to one embodiment of the invention the substantially flexible longitudinal portion is coated with a substantially biocidal material. In one embodiment, the substantially biocidal material includes a metallic silver material. In one embodiment, the metallic silver material comprises a 99.99% pure metallic silver region. In still another embodiment of the invention, the metallic silver material comprises a 99.995% pure metallic silver region. In one embodiment of the invention, the metallic silver material includes a metallic silver material having a purity in a range from about 99.99% to about 99.995% pure. According to one embodiment of the invention, the substantially biocidal material is disposed in a substantially uniform thickness about a profile of the substantially flexible longitudinal portion 104.

According to one embodiment of the invention, a metallic biocidal material is combined with a further biocidal material such as, for example, one or more of an antibiotic material, an antiseptic material an antiviral material and a fungicidal material. The practitioner of ordinary skill in the art will understand that a wide variety of such biocidal materials can be applied in various embodiments and, for example, captured in grooves of fibers, as illustrated in FIG. 2. For example, and without intending to provide a comprehensive, such biocidal materials include Acyclovir (Zovirax®), Amantadine (Symmetrel®), Aminoglycosides, Amoxicillin (generic), Amoxicillin/Clavulanate (Augmentin®), Amphotericin B, nonlipid (Fungizone®), Ampicillin (generic), Ampicillin/sulbactam (Unasyn®), Atovaquone (Mepron®), Azithromycin (Zithromax®), Cefazolin (generic), Cefepime (Maxipime®), Cefotaxime (Claforan®), Cefotetan (Cefotan®), Cefpodoxime (Vantin®), Ceftazidime (generic), Ceftizoxime (Cefizox®), Ceftriaxone (Rocephin®), Cefuroxime (Zinacef®), Cephalexin (generic), Chloramphenicol (generic), Clotrimazole (Mycelex®), Ciprofloxacin+(Cipro®), Clarithromycin+(Biaxin®), Clindamycin+(Cleocin®), Dapsone, Dicloxacillin (generic), Doxycycline (generic), Erythromycin lactobionate+(generic), Fluconazole+(Diflucan®), Foscamet (Foscavir®), Ganciclovir (Cytovene® DHPG), Gatifloxacin (Tequin®), Imipenem/Cilastatin (Primaxin®), Isoniazid (generic), Itraconazole+(Sporanox®), Ketoconazole+(generic), Metronidazole+, Nafcillin, Nitrofurantoin, Nystatin (generic), Penicillin G (generic), Pentamidine (generic), Piperacillin/Tazobactam (Zosyn®), Rifampin+(Rifadin®), Quinupristin-Dalfopristin (Synercid®), Ticarcillin/clavulanate (Timentin®), Trimethoprim sulfamethoxazole (generic), Valacyclovir (Valtrex®), Vancomycin (generic), and combinations thereof. In a further embodiment, any other appropriate medical or pharmaceutical agent can be disposed on the surface of the wound closure device.

According to one embodiment of the invention, a metallic silver material is deposited on the substantially flexible longitudinal portion 104 according to a deposition process described in U.S. Pat. No. 7,172,785 (hereinafter the '785 patent) issued Feb. 6, 2007 to G. Alan Thompson, et al., the disclosure of which is herewith incorporated by reference in its entirety. According to one aspect of the invention, a suture including a metallic needle is provided with a metallic coating about a longitudinal portion thereof, by immersing a region of the longitudinal portion in a fluid while maintaining the metallic needle outwardly of the fluid. According to certain embodiments of the invention, ultrasonic energy is applied to the suture during processing to facilitate a deposition of metallic silver material from the processing fluid onto an external surface of the suture. According to one embodiment of the invention, an additional chemical burnishing is applied to the longitudinal portion subsequent to application of the metallic coating.

In one embodiment of the invention, a portion of a needle is disposed within a substantially elastic medium during a deposition process according to the '785 patent, whereby the needle is appropriately fixtured while the substantially flexible longitudinal portion 104 is disposed within a processing fluid. In one embodiment of the invention, the substantially elastic medium includes a natural polymer material such as, for example, a natural rubber material. In another embodiment of the invention, the substantially elastic medium includes a synthetic polymer materials such as, for example, a neoprene material, and in one example a neoprene closed cell foam material. In another embodiment, the substantially elastic medium includes a polyethylene foam material. In still another embodiment of the invention, the substantially elastic medium includes a cellulose material such as, for example, a cork material.

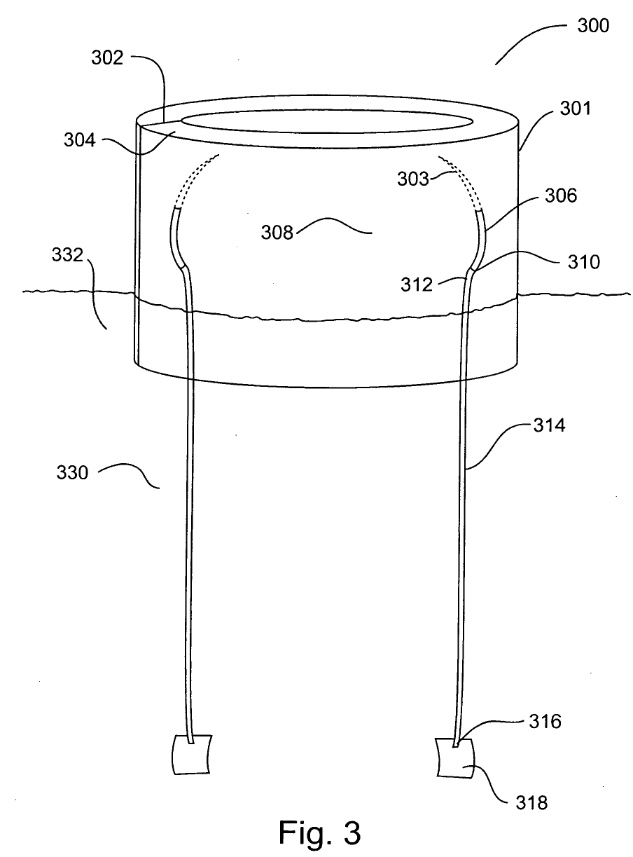

FIG. 3 shows one arrangement for manufacturing a suture according to principles of the invention. As illustrated in FIG. 3, a fixturing device 300 includes a support member 301 incorporating a material adapted to receive one or more needles 306 substantially elastically therewithin. Thus, as illustrated, a point 303 of a needle is lodged through a surface 308 and within a bulk region of support member 301. An opposite end 310 of needle 306 is coupled to a proximate end 312 of a suture 314. A distal end 316 of suture 314 is coupled, in one embodiment, to a weighted device 318 so that suture 314 is held in a substantially vertical orientation depending from needle 306.

In certain embodiments of the invention, a clamp or clip mechanism is used to support the needle, rather than, or in combination with, having the tip of the needle 303 embedded in the material of the support member 301. In another embodiment of the invention, a plurality of sutures 314 are mutually coupled at respective distal ends 316 to a common weighted device 318. In certain embodiments, the weighted device includes a polymer material such as, or example, a polyvinyl chloride material. In still another embodiment of the invention, an extended more or less rigid member is coupled to distal end 316 of the suture, rather than extending the suture by weighting it.

In one exemplary embodiment, as noted above, the material of the support member 301 includes a cork material. Thus, in the illustrated embodiment, the fixturing device 300 includes a sheet of cork material formed into a curve about a longitudinal axis and coupled at adjacent edges 302, 304 to form a substantially cylindrical support member.

In one embodiment of the invention, as illustrated in FIG. 3, the support member 301 is disposed substantially buoyantly in a processing solution 330. In another embodiment of the invention, the support member is suspended above an upper surface 332 of the processing solution. One of skill in the art will appreciate that in certain embodiments of the invention, deposition of a metallic silver material on the suture 314 is a achieved by moving the support member 301, along with needles and sutures coupled thereto, successively from one bath of processing solution to another bath of processing solution until processing is complete.

One of skill in the art will also appreciate that, in other embodiments of the invention, a continuous processing methodology is employed to produce a silverized suture material, either in continuous runs or in precut lengths. Subsequently, the silverized suture material is coupled to needles by any of the various methods discussed below. In still other embodiments of the invention, a polymer piercing device, or needle, is coupled to the suture and is immersed in the processing fluid so that the polymer piercing device is metallized along with the suture material.

Making further reference to FIG. 1, in various embodiments of the invention, a coupling between the first end 106 of the longitudinal portion 104 and the second end 108 of the needle 102 is effected by a swaging process. According to one embodiment, the second end 108 of the needle 102 includes an internal surface defining a longitudinal cavity. The first end 106 of the longitudinal portion 104 is disposed within the longitudinal cavity, and a portion of the needle 102 is compressed to bring the internal surface of the needle 102 into intimate contact with an external surface of the first end 106, whereby the longitudinal portion is operatively affixed to the needle.

In another embodiment of the invention, a coupling between the needle 102 and the longitudinal portion 104 is achieved by a process of disposing a portion of the longitudinal portion within and through an eye of the needle, as is known in the art. According to one embodiments of the invention, a compression of the needle is employed to reduce a dimension of the eye and thereby retain the portion of the longitudinal portion within the eye of the needle. In still another embodiment of the invention, an adhesive is applied between the longitudinal portion and the needle to maintain a coupling between the longitudinal portion and the needle. In still another embodiment of the invention, a thermal welding process is employed to establish a coupling between the longitudinal portion 104 and the needle 102. In yet another embodiment of the invention, an ultrasonic welding process is employed to establish a coupling between the longitudinal portion 104 and the needle 102. In yet another embodiment of the invention, an electrochemical deposition is employed to establish a mutual metallic bond between a silverized surface of the longitudinal portion 104 and the needle 102.

According to one embodiment of the invention, the longitudinal portion 104 includes a material that is substantially non-reactive and non-absorbable in the environment of a patient's body. Accordingly, subsequent removal of the non-absorbable suture allows a corresponding removal of the remaining metallic material disposed on and/or within the suture. An important and nonobvious benefit of this removability is that while silver ions are able to diffuse into surrounding tissue for their biocidal effect, substantially no bulk silver is left within the body of the patient after removal of the suture. This avoids the pigmentation that can be experienced in association with a deposition of bulk silver within a body, and other possible effects of the presence of remaining bulk silver.

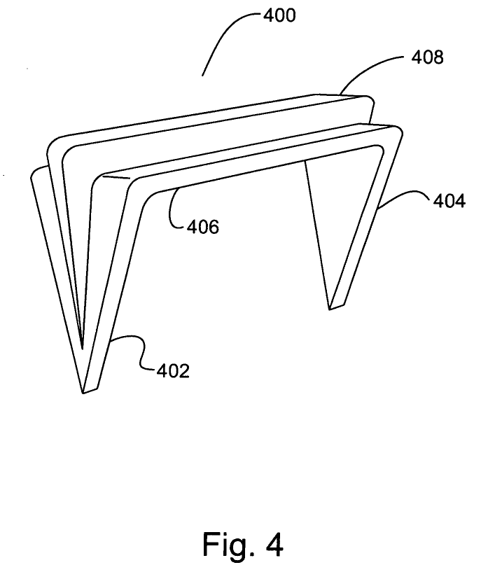

In another embodiment of the invention, as shown in FIG. 4, a wound closure device includes a surgical staple 400. According to one embodiment of the invention, the surgical staple 400 includes a first needlelike penetrating portion 402, and a second needlelike penetrating portion 404. A longitudinal portion 406 is disposed between the first penetrating portion 402 and the second penetrating portion 404. According to one embodiment of the invention, as illustrated, a flange portion 408 is disposed to provide rigidity to the first 402 and second 404 penetrating portions and the longitudinal portion 406.

According to one embodiment, the staple 400 includes a stainless steel material including, for example, a surgical stainless steel. According to a further embodiment of the invention, the staple portion 400 includes a rigid polymer material. In one embodiment, the stainless steel material is substantially entirely enclosed within the polymer material. In another embodiment of the invention, the stainless steel material is disposed adjacent to a surface of the polymer material. In still other embodiments, no metallic portion is disposed within the polymer material. In various embodiments, the synthetic polymer material includes a polyamide material, a polyaramid material, a polybutylene material; an acrylonitrile butadiene styrene (ABS) polymer material, a polypropylene material, a polyvinyl chloride material, a polyester material, or other synthetic and natural polymer material, including combinations thereof, as known in the art.

According to one embodiment of the invention the staple 400 is coated with a substantially biocidal material. In one embodiment, the substantially biocidal material includes a metallic silver material. In one embodiment, the metallic silver material comprises a 99.99% pure metallic silver region. In still another embodiment of the invention, the metallic silver material comprises a 99.995% pure metallic silver region. In one embodiment of the invention, the metallic silver material includes a metallic silver material having a purity measurable in a range from about 99.99% to about 99.995%. According to one embodiment of the invention, the substantially biocidal material is disposed in a substantially uniform thickness about a profile of the staple 400. In another embodiment of the invention, a further biocidal material is included on a surface of the staple or disposed within pores in the surface of the staple. According to certain embodiments of the invention, the further biocidal material includes one or more of an antibiotic and an antiseptic.

In one embodiment, the invention includes a stapling device adapted to deposit a portion of the staple 400 through a region of a patient's tissue adjacent to a wound, whereby the staple 400 is adapted to retain the wound in a substantially closed arrangement. According to one embodiment, the stapling device is adapted to fold the first 402 and second 404 penetrating portions with respect to the longitudinal portion 406 so as to substantially fix the staple 400 in place, with respect to the surrounding skin. In one embodiment of the invention, the stapling device is adapted to deposit a further biocidal material onto a surface of the patient's skin or flesh in a location where the staple 400 will penetrate. In certain embodiments, the deposited biocidal material is deposited in a liquid form, a gel form, a paste form, a powder form, a gaseous form, or any other form adapted to effectively reach and destroy pathological agents disposed in the region of penetration.

According to one embodiment, the stapling device includes a disposable stapling device, and the invention includes a method of providing a disposable stapling device which can be disposed of once a pre-loaded charge of staples is expended. In another embodiment of the invention, a rechargeable stapling device is provided, and in still another embodiment of the invention, a disposable stapling device is provided with a rechargeablity feature such that the stapling device may be used as either a disposable or a rechargeable stapling device.

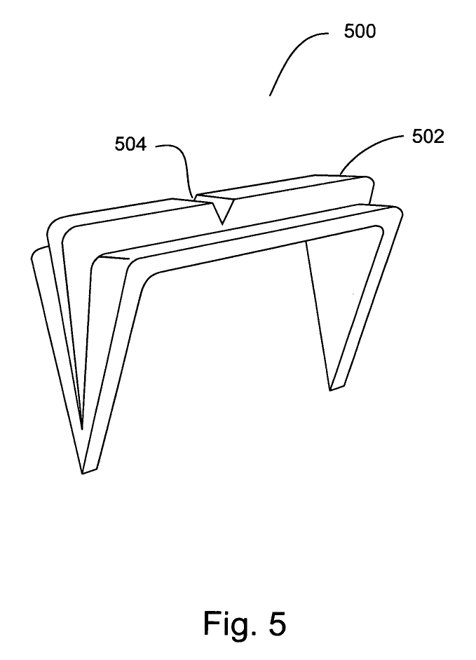

FIG. 5 shows a further embodiment of the invention including a wound closure device 500. According to one embodiment of the invention, the wound closure device 500 includes a staple having a bridge top 502. The bridge top 502 has a notch 504 in an upper surface thereof. The notch 504 is sized and positioned to permit a practitioner to readily bend, cut or break the staple 504 removal after use of the staple is complete. In one method, according to principles of the invention, a removal tool is used to grasp, lift and cut or fracture the staple adjacent to the notch during staple removal. Also included in the invention is a removal device, such as a pliers device, adapted to remove the staple device. According to one embodiment of the invention, the removal pliers include a disposable removal pliers device.

According to one embodiment, the invention includes a surgical drain device. An exemplary surgical drain device includes a nonreactive synthetic material impregnated with a silver material. According to one embodiment of the invention the surgical drain device provides immediate and sustained antimicrobial action in surgical wounds requiring a drain by virtue of the antimicrobial properties of the silver material. According to one aspect of the invention, the surgical drain device can subsequently be removed, whereby substantially all bulk silver is removed from the body of the patient. According to one embodiment of the invention, the surgical drain device comprises a longitudinal portion like portion 104 of FIG. 1, the longitudinal portion having a substantially hollow internal longitudinal region.

According to certain embodiments of the invention, a staple device, such as wound closure device 500 of FIG. 5, is produced by injection molding according to methods known in the art. In certain embodiments of the invention, a plurality of staple devices are prepared including coupling members between the devices, so that chemical processing to provide silverization according to the methods discussed above can be substantially simultaneously performed on the plurality of staple devices. Thereafter, the plurality of staple devices can be separated from one another and the connecting members can be discarded or reprocessed and recycled.

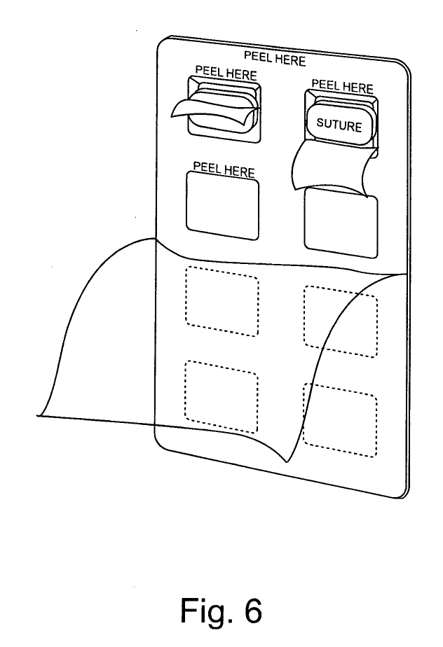

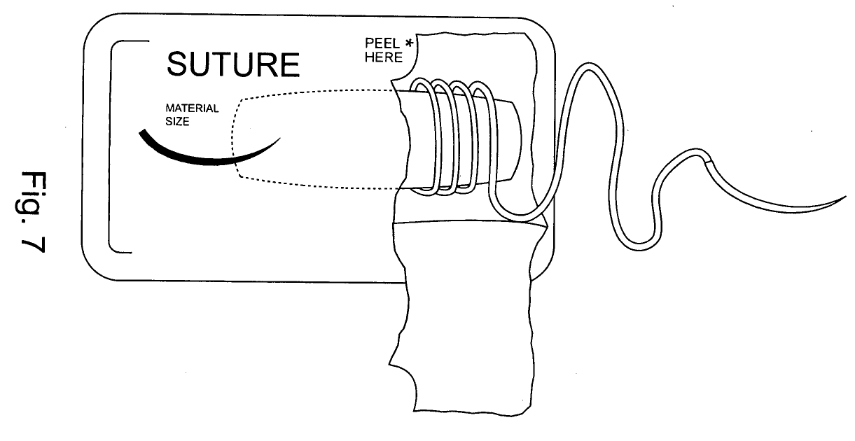

According to one embodiment of the invention the devices described hereinabove are provided in substantially sterile packaging in single unit packages, and in multiple device packages. In one embodiment, the packaging includes a non-sterile outer shell having an easy-tear notch and an internal protective sterilized sleeve, pouch or envelope. According to one embodiment of the invention, color coding is applied to a package according to, and indicating, a presence of a biocidal material incorporated into a wound closure device disposed within the package. One exemplary multi-unit package according to the invention is shown in FIG. 6. A further exemplary single-unit packet according to the invention is shown in FIG. 7.

In certain embodiments of the invention, sterilization of the wound closure device is accomplished by irradiating the device with, for example, gamma radiation, or radiation of other effective wavelengths, after the wound closure device is enclosed in a sealed package. In other embodiments of the invention, the wound closure device is sterilized by chemical or thermal processing prior to packaging in a sterile package. For example in one embodiment of the invention, the wound closure device is heated in an autoclave prior to packaging.

In one embodiment of the invention, a wound closure device, according to one or more of the various embodiments described above, is adapted to be employed in the treatment of a human patient. In another embodiment of the invention, a wound closure device, according to one or more of the various embodiments described above, is adapted to be employed in the treatment of a non-human (veterinary) patient.

Without wishing to be bound to a theory of operation, it is believed that during use, bodily fluids including blood plasma contact the surface of the wound closure device. Consequently, biocidal material, such as ions of silver, are conveyed from the surface of the wound closure device to surrounding tissue, so as to impede or kill pathogens introduced, for example, by installation of the wound closure device. According to one embodiment of the invention, deep group fibers are particularly effective in bringing bodily fluids into contact with the biocidal material by capillary action. According to a further aspect of the invention, the described silverization process is particularly effective at providing silver within the deep grooves of deep-groove fibers.

While the exemplary embodiments described above have been chosen primarily from the field of emergency medicine, one of skill in the art will appreciate that the principles of the invention are equally well applied, and that the benefits of the present invention are equally well realized in a wide variety of other medical applications including, for example, elective and non-elective surgeries, as well as both human and veterinary medical applications. Further, while the invention has been described in detail in connection with the presently preferred embodiments, it should be understood that the invention is not limited to such disclosed embodiments. Rather, the invention can be modified to incorporate any number of variations, alterations, substitutions, or equivalent arrangements not heretofore described, but which are commensurate with the spirit and scope of the invention. Accordingly, the invention is not to be seen as limited by the foregoing description, but is only limited by the scope of the appended claims.

1. A wound closure device comprising:
a piercing portion;
a longitudinal portion having a first end coupled to said piercing portion, said longitudinal portion having a region with a deep groove profile and a biocidal metallic coating.

2. A wound closure device as defined in claim 1 wherein said biocidal metallic coating comprises a substantially uniformly distributed biocidal metallic coating.

3. A wound closure device as defined in claim 1 wherein said biocidal metallic coating comprises a substantially pure metallic silver material.

4. A wound closure device as defined in claim 3 wherein said substantially pure metallic silver material comprises a metallic silver material having a purity from at least about 99.9% to at least about 99.999% pure.

5. A wound closure device as defined in claim 3 wherein said substantially pure metallic silver material comprises a metallic silver material having a purity from at least about 99.99% to at least about 99.995% pure.

6. A wound closure device as defined in claim 1 further comprising a further biocidal material disposed at a surface thereof.

7. A wound closure device as defined in claim 6 wherein said further biocidal material comprises an antibiotic material.

8. A wound closure device as defined in claim 6 wherein said further biocidal material comprises an antiseptic material.

9. A wound closure device as defined in claim 6 wherein said further biocidal material comprises an antiviral material.

10. A wound closure device as defined in claim 6 wherein said further biocidal material comprises a fungicidal material.

11. A wound closure device as defined in claim 1 wherein said longitudinal portion having a region with a deep groove profile comprises a high capillary-action fiber material.

12. A wound closure device as defined in claim 1 wherein said piercing portion comprises a stainless steel material.

13. A wound closure device as defined in claim 1 wherein said piercing portion comprises a metallized polymer material.

14. A wound closure device as defined in claim 13 wherein said metallized polymer portion comprises a reinforced polymer portion.

15. A wound closure device as defined in claim 14 wherein said reinforced polymer portion comprises a carbon reinforced polymer portion.

16. A wound closure device as defined in claim 15 wherein said carbon reinforced polymer portion comprises a carbon nanotube reinforced polymer portion.

17. A wound closure device as defined in claim 14 wherein said reinforced polymer portion comprises a glass fiber reinforced polymer portion.

18. A wound closure device as defined in claim 13 wherein said metallized polymer portion comprises a silverized polymer portion.

19. A method of closing a wound comprising:
providing a wound closure device having a silverized polymer portion;
piercing tissue adjacent to a wound to form a hole in the tissue;
disposing the silverized polymer portion of the wound closure device within the hole in the tissue;
disposing the silverized polymer portion of the wound closure device in tension so as to urge closure of the wound; and
allowing silver ions to diffuse from the silverized polymer portion into the tissue so as to provide a biocidal action against pathogens within the tissue.

20. A method of closing a wound comprising:
providing a wound closure device having a silverized polymer portion, said silverized polymer portion including a silverized region having a purity of from at least about 99.99% to at least about 99.995% pure;
piercing tissue adjacent to a wound with the wound closure device to form a hole in the tissue;
disposing the silverized polymer portion of the wound closure device within the hole in the tissue;
disposing the silverized polymer portion of the wound closure device in tension so as to urge closure of the wound; and
allowing silver ions to diffuse from the silverized polymer portion into the tissue so as to provide a biocidal action against pathogens within the tissue.

21. A method of closing a wound comprising:
providing a wound closure device having a silverized polymer portion, said silverized polymer portion including a silverized region having a purity of from at least about 99.99% to at least about 99.995% pure, said silverized region being disposed within a deep groove of said wound closure device;
piercing tissue adjacent to a wound with the wound closure device to form a hole in the tissue;
disposing the silverized polymer portion of the wound closure device within the hole in the tissue;
disposing the silverized polymer portion of the wound closure device in tension so as to urge closure of the wound; and
allowing silver ions to diffuse from the silverized polymer portion into the tissue so as to provide a biocidal action against pathogens within the tissue.

22. A method of closing a wound as defined in claim 21 wherein said deep groove comprises a groove having an aspect ratio of from at least about 50% to at least about 10%.

23. A method of closing a wound as defined in claim 21 wherein said deep groove comprises a groove having an aspect ratio of from at least about 40% to at least about 1%.
